(19) United States
(12) Patent Application Publication
(10) Pub. No.: US 2019/0307499 A1
(43) Pub. Date: Oct. 10, 2019

(54) ABLATION DEVICE HAVING A SHEATH WITH A DILATABLE MEMBER FOR FIXATION AND/OR SUPPORT OF AN ABLATION APPLICATOR, AND METHOD OF OPERATING AN ABLATION DEVICE

(71) Applicant: AFREEZE GMBH, Innsbruck (AT)

(72) Inventors: Florian HINTRINGER, Ampass (AT); Gerald FISCHER, Völs (AT)

(21) Appl. No.: 16/307,889

(22) PCT Filed: Jun. 7, 2017
(86) PCT No.: PCT/EP2017/063884

§ 371 Date: Dec. 6, 2018

(30) Foreign Application Priority Data
Jun. 8, 2016 (EP) 16173515.4

(51) Int. Cl. A61B 18/00

(52) U.S. Cl. CPC A61B 18/00; A61B 2018/0212; A61B 2018/00577; A61B 2018/00285

(57) ABSTRACT

An ablation device is described. The ablation device comprises (a) an ablation catheter having an ablation applicator, and (b) a sheath comprising an elongate tubular member and a dilatable member arranged at the distal end of the elongate tubular member. The elongate tubular member defines an inner lumen adapted to receive the ablation catheter having the ablation applicator, wherein the elongate tubular member comprises an opening at a distal end thereof, the opening being adapted to allow the ablation applicator to extend in front of the distal end. The elongate tubular member comprises an outer wall in which a canal structure is formed, wherein the canal structure is in fluid communication with the dilatable member, and wherein the canal structure is adapted to supply fluid to the dilatable member in order to dilate it and to discharge fluid from the dilatable member in order to compress it. The ablation catheter is slidably arranged within the inner lumen of the sheath. Furthermore, an ablation system and an ablation method are described.

### CROSS-REFERENCE TO RELATED APPLICATIONS

This application is a National Phase Patent Application of International Patent Application Number PCT/EP2017/063884, filed on 7 Jun. 2017, which claims priority of European Patent Application No. 16173515.4 filed on 8 Jun. 2016, the disclosures of which are hereby incorporated herein by reference.

### TECHNICAL FIELD

Embodiments of the invention relates to the field of medical devices, more specifically to ablation devices and to a method of operating an ablation device.

### TECHNOLOGICAL BACKGROUND

There exists a wide variety of devices and systems for removing abnormal or diseased tissue by ablation in order to treat various medical conditions. In many cases, ablation is preferable to conventional surgery as it is usually less invasive. In some cases, conventional surgery may not even be possible. Important applications of ablation are e.g. the treatment within blood vessels, e.g. within or in the vicinity of the human heart or kidneys.

Generally, when using ablation techniques such as cryoablation (application of extreme cold), heat ablation and RF (radio frequency) ablation, where an ablation applicator is brought into direct contact with the tissue that is to be treated, it is important to keep the ablation applicator in place and in firm contact with the tissue during the treatment. Furthermore, it may be important to provide isolation between the ablation site and surrounding tissue and blood during the ablation process.

Balloon ablation catheters can be used to treat atrial fibrillation by ablation at the ostium of a pulmonary vein in the left atrium of the heart. Here, the ablation applicator is incorporated inside a balloon which is used for fixing the device at the ostium of the vein. A known cryoablation balloon catheter is described in U.S. Pat. No. 6,575,966 B2. Balloon ablation catheters are restricted to treat the tissue essentially at the contact area of the balloon with the tissue. Generally, this contact area is inside the ostium of the vein leaving tissue in the antrum of the vein untreated. Balloon ablation catheters provide no mechanism which allows shifting the ablation structure relative to the area of fixation.

A loop shaped catheter can be used for ablation inside the ostium of the vein and in the antrum of a vein. DE 103 18 478 A1 describes a loop shaped ablation structure with a balloon mounted proximally from the loop on the same catheter body. The loop may be used for pressing the applicator against the tissue, thereby increasing contact force. However, as both structures are mounted on the same device, the movement of the structures relative to each other is severely limited. Formation of the loop requires pulling the applicator structure away from the tissue. Due to the restriction in the relative movement of both structures it might be difficult to effectively shape the ablation applicator while pressing it against the tissue.

DE 102 18 427 A1 describes a loop shaped ablation applicator with a loop on the distal end of the same catheter device. Also for this device, the movement of the ablation structure relative to the fixation structure is limited, which might hamper optimal wall contact.

### SUMMARY

There may be a need for a way of enabling a good contact between an ablation applicator and tissue and for a way of enabling efficient isolation against surrounding tissue and/or blood during ablation, in a simple and cost-efficient manner.

This need may be met by the ablation devices and the method of operating an ablation device set forth in the independent claims.

According to an exemplary embodiment, an ablation device is provided, the ablation device comprising an ablation catheter having an ablation applicator, and a sheath comprising an elongate tubular member and a dilatable member arranged at the distal end of the elongate tubular member, wherein the elongate tubular member defines an inner lumen adapted to receive the ablation catheter having the ablation applicator, wherein the elongate tubular member comprises an opening at a distal end thereof, the opening being adapted to allow the ablation applicator to extend in front of the distal end, wherein the elongate tubular member comprises an outer wall in which a canal structure is formed, wherein the canal structure is in fluid communication with the dilatable member, and wherein the canal structure is adapted to supply fluid to the dilatable member in order to dilate it and to discharge fluid from the dilatable member in order to compress it, and wherein the ablation catheter is slidably arranged within the inner lumen of the sheath.

According to an alternative exemplary embodiment, an ablation device is provided, the ablation device comprising an ablation catheter having an ablation applicator, and a sheath comprising an elongate tubular member and a dilatable member arranged at the distal end of the elongate tubular member, wherein the elongate tubular member defines an inner lumen adapted to receive the ablation catheter having the ablation applicator, wherein the elongate tubular member comprises an opening at a distal end thereof, the opening being adapted to allow the ablation applicator to extend in front of the distal end, wherein the sheath further comprises a mechanical control mechanism operable to dilate the dilate the dilatable member and to compress it, and wherein the ablation catheter is slidably arranged within the inner lumen of the sheath.

According to another exemplary embodiment, an ablation system is provided, the ablation system comprising an ablation device having the above-mentioned features, a fluid source, and a pump for supplying fluid to the dilatable member and for discharging fluid from the dilatable member.

According to still another exemplary embodiment, a method of operating an ablation device having the above mentioned features is provided, the method comprising positioning the ablation catheter and sheath in an object, dilating the dilatable member by supplying a fluid to the dilatable member through the canal structure, operating the ablation device to perform an ablation process, discharging the fluid from the dilatable member, and removing the ablation device from the object.

According to still another exemplary embodiment, a method of operating an ablation device having the above mentioned features is provided, the method comprising positioning the ablation catheter and sheath in an object, dilating the dilatable member by operating the mechanical control mechanism, operating the ablation device to perform an ablation process, compressing the dilatable member by operating the mechanical control mechanism, and removing the ablation device from the object.

The term “ablation device” may particularly denote any apparatus which is adapted to ablate, deactivate, destroy or remove material, particularly tissue of a physiological object such as a human being or an animal, via the application of an ablation medium, such as extreme cold or heat, radio frequency (RF) electric current, a laser beam, etc. . . .

The term “ablation catheter” may particularly denote a catheter, i.e. a tube that can be inserted into a body cavity, duct or vessel in order to conduct an ablation treatment. Ablation catheters may thereby allow access by surgical instruments. An ablation catheter is a part of an ablation device and may be a flexible tube-like unit comprising one or more internal lumens and connections, e.g. for transporting or guiding an ablation medium to and from the point or area of application, for steering and guiding the catheter, for communicating sensor data, etc. In other embodiments, an ablation catheter may be a stiff tube-like unit. Its diameter may in particular vary between 0.3 mm and 10 mm.

The term “ablation applicator” may particularly denote a part for applying the ablation energy or medium to the relevant tissue. The ablation applicator may in particular be a dedicated part of an ablation catheter, such as a tip of the catheter, or a separate part connected with the ablation catheter.

The term “dilatable member” may particularly denote a member or part that can increase and decrease in size, e.g. that can be expanded like a balloon when it its filled with a fluid and compressed by discharging the fluid. The dilatable member may particularly be made of a material with elastic properties. In particular, a compliant balloon material, such as for example a polyurethane, a nylon elastomer or another thermoplastic elastomer may be used. Materials of a high compliance range might be chosen which can be inflated at low pressures. Preferably, the material of the dilatable member has a dilation range of at least 100%, such as around 200% or even more.

According to an exemplary embodiment, an ablation device comprises a sheath having an inner lumen (within an elongate tubular member) for receiving an ablation catheter of an ablation device is provided with a dilatable member at the distal end of the elongate tubular member. The dilatable member is in fluid communication with a canal structure provided within an outer wall of the elongate tubular member. Thereby, a fluid, such as a saline solution or a mixture of saline solution and a contrast agent or any other suitable fluid which does not harm a patient in case of a leakage and which, in the case of cryoablation, may provide some or a significant frost protection, can be supplied to the dilatable member and discharged from the dilatable member in order to respectively dilate and compress the dilatable member.

The ablation catheter is slidably arranged within the inner lumen of the sheath. Thereby, once the ablation catheter has been brought into position such that the ablation applicator is correctly positioned relative to the tissue that is to be ablated, the sheath may be moved relative to the ablation catheter to optimize the position of the dilatable member and/or to assure that the dilatable member applies a pressing force on the ablation applicator, in particular in the axial direction of the sheath.

More specifically, once the sheath (together with an ablation catheter arranged within the inner lumen) is brought into the position (e.g. within a cavity such as one of the cardiac chambers, or a blood vessel) where ablation is to be performed, i.e. such that the ablation applicator extending in front of the sheath and ablation catheter is correctly positioned relative to the tissue that is to be ablated, the fluid is supplied through the canal structure in the outer wall of the sheath in order to dilate the dilatable member. Depending on the location within the body (in particular size and shape of cavity or vessel) and the size and shape of the dilatable member, the expansion of the dilatable member may provide one or more of the following advantageous effects: (i) improved fixation of sheath and ablation catheter in the desired position, (ii) application of a pressing force on the ablation applicator, thereby e.g. improving contact with tissue to be ablated, and (iii) acting as an isolating element between the ablation applicator and surrounding tissue and/or blood, thereby concentrating the ablation effects to the desired area(s) while protecting the surroundings and/or avoiding that warm blood from the surroundings flows towards the ablation site.

The third effect, i.e. the isolation, is particularly advantageous when ablation is performed by application of heat or cold, i.e. in connection with cryosurgery, which is the application of extreme cold to ablate abnormal or diseased tissue. Cryosurgery works by taking advantage of the destructive force of freezing temperatures on cells. At low temperatures, ice crystals may form inside the cells, which can tear them apart. More damage may occur when blood vessels supplying the tissue freeze.

Next, further exemplary embodiments of the ablation device will be explained and discussed. However, these embodiments also apply to the alternative ablation device, to the ablation system and to the methods.

The canal structure may comprise a supply canal and a discharge canal. In other words, the canal structure may comprise one canal (the supply canal) for supplying fluid to the dilatable member and another canal (the discharge canal) for discharging fluid from the dilatable member.

At least one of the supply canal and the discharge canal, i.e. the supply canal and/or the discharge canal, may be formed as a tubular canal extending within the outer wall in the axial direction of the sheath. In other words, at least one tubular canal (e.g. with a substantially circular or substantially elliptic cross-sectional shape) may extend within the outer wall in the length dimension of the sheath.

Alternatively, or additionally, at least one of the supply canal and the discharge canal may be formed as a substantially rectangular canal extending within the outer wall in the axial direction of the sheath. In other words, at least one canal with a substantially rectangular cross-sectional shape may extend within the outer wall in the length dimension of the sheath. In this context, the term “substantially rectangular” is intended to include any cross-sectional shape having two pairs of substantially parallel side walls.

The canal structure may comprise a plurality of supply canals and/or a plurality of discharge canals. In other words, the canal structure may comprise more than one supply canal and/or more than one discharge canal. Each of these canals may have any of the features discussed above. For example, the plurality of supply canals and/or the plurality of discharge canals may extend in parallel in the axial direction of the sheath.

At least one of the supply canal and the discharge canal may be formed as an opening extending radially through the sheath, thereby bringing the dilatable member in fluid communication with the inner lumen. In this case, the opening allows fluid transport between the dilatable member and the inner lumen. In other words, to e.g. fill the dilatable member with fluid (such as a saline solution with or without a contrast agent), the fluid may be supplied into the inner lumen of the sheath at a proximate end of the sheath and stream through the inner lumen and the opening into the dilatable member. Similarly, fluid may be withdrawn or discharged from the dilatable member by applying a negative pressure (i.e. by suction) at the proximate end of the sheath such that the fluid is transported from the distal member, through the opening and out through the inner lumen of the sheath.

The dilatable member may be arranged at a distal end section of the outer wall. In particular, the dilatable member may be surrounding the distal end section of the outer wall. In other words, the dilatable member may be arranged at or around the outside of the distal part (such as the last 1 to 5 cm) of the sheath. In its empty, i.e. not dilated state, the dilatable member preferably fits closely around the sheath without causing any significant increase in the diameter of the distal end section of the sheath. Thus, the dilatable member does not restrict or hinder the insertion of the sheath into narrow openings, cavities or vessels of an object, such as a human being or an animal. When the dilatable member is filled by fluid and thus dilated, it increases the effective diameter of the sheath (in a symmetric or asymmetric manner depending on the shape of the dilatable member).

Alternatively, the dilatable member may be arranged at a distal end section or at a distal tip of an inner wall of the elongate tubular member. By arranging the dilatable member at an inner wall of the elongate tubular member, i.e. within the inner lumen of the sheath, the dilatable member—as long as it is not filled with fluid—will (at least partially) be contained within the sheath, such that the sheath (with ablation catheter) will be particularly easy to insert and guide towards the ablation site. Furthermore, if desirable, it may be easier to obtain a configuration where the dilatable member—in the dilated state where it is filled with fluid—extends to some extent in front of the distal end of the sheath. In this case, the dilatable member may in particular be used to apply a radial pressing force towards an ablation applicator, in particular when the ablation applicator is shaped like a loop or helix surrounding the dilatable member. The latter applies equally to the alternative configuration, where the dilatable member is arranged at the distal tip.

The dilatable member may, in its dilated state, have a shape selected from the group consisting of a torus, a torus section, a sphere, a sphere section, a cylinder, a cone section, and a pear-like shape. These various shapes have individual advantages and properties depending inter alia on the geometry of the ablation site and on the desired effect(s) to be obtained. A torus (section), a sphere (section) and a pear-like shape may e.g. be advantageous with regard to fixation of the sheath (and an ablation catheter therein), with regard to applying an axial pressing force on an ablation applicator in front of the distal end of the sheath, and with regard to isolating surrounding tissue and blood from the ablation applicator. On the other hand, the shapes of a cylinder or a cone section may be more suitable in situations where it is desirable that the dilatable member extends in front of the distal end of the sheath, e.g. in order to apply a radial pressing force on an ablation applicator. In the latter case, the dilatable member may extend within a loop or helix shaped ablation applicator and thereby apply a radial (outward) pressing force on the applicator.

The ablation device may furthermore comprise at least one temperature sensor for monitoring the temperature in the vicinity of the ablation applicator.

The at least one temperature sensor may by arranged within the dilatable member in a position close to the ablation applicator. Alternatively, the at least one temperature sensor may be located within the sheath, within the ablation catheter, within the ablation applicator, and/or within a tip of the ablation catheter.

The at least one temperature sensor enables monitoring of the temperature in the vicinity of the ablation applicator. Thereby, an operator may e.g. be able to determine whether the fluid inside the dilatable member is frozen, in which case the sheath and/or the dilatable member should not be moved or withdrawn.

The ablation applicator may be selected from the group consisting of a cryoablation applicator, a heat ablation application, a radio frequency electrical ablation applicator, a microwave ablation applicator, and a laser ablation applicator.

The dilatable member may, as also mentioned above, be adapted to, in a dilated state, apply a pressing force on the ablation applicator, in particular to apply a pressing force on the ablation applicator in an axial or radial direction of the sheath.

Additionally or alternatively, as also mentioned above, the dilatable member may be adapted to, in a dilated state, act as an isolating element between the ablation applicator and surrounding tissue and/or fluid, in particular blood.

The ablation catheter, which is slidably arranged within the sheath and thus movable relative to the sheath, may comprise a closing member arranged to block fluid communication between the inner lumen of the sheath and at least a part of the canal structure when the ablation catheter and the sheath are in a predetermined positional relationship. In particular, the closing member may be a protrusion on the outer surface of the ablation catheter. For example, the protrusion may extend partially around the perimeter of the ablation catheter and be adapted to block a hole extending in a radial direction through the sheath, when the ablation catheter is correspondingly positioned relative to the sheath. The protrusion may also extend completely around the perimeter of the ablation catheter, thereby forming a ring-shaped gasket which, when located on the distal side of a hole extending in a radial direction through the sheath, allows fluid communication between the inner lumen and the hole while, when located on the proximate side of the hole, blocks fluid communication between the inner lumen and the hole.

The ablation applicator may have a longitudinal curved shape. In particular, the ablation applicator may have a shape selected from the group consisting of a loop shape and a helix shape.

The application applicator may be made from a shape memory material such as Nitinol, or copper or iron based alloys. They will be designed such that they display superelastic properties at room and body temperatures. Here super- or pseudoelastic means that the material can withstand a relatively large reverse geometrical deformation. Thus the material can be brought into a stretched position for insertion into the body and can be moved to an organ such as the heart along curved vessels. At the organ the material can be released to its original predetermined shape such as a loop or helix or an arbitrary curved longitudinal shape. In embodiments where the ablation applicator is part of a cryo-ablation catheter, the shape memory material will be cooled to low temperatures. This will trigger a change of the material from austenite to martensitic phase. For maintaining shape and increasing stiffness of the material at body temperature and initial cooling, a shape memory metal with a low transition temperature, such as chrome dotted nitinol, may be used. Next, further exemplary embodiments of the alternative ablation device will be explained. However, these embodiments also apply to the ablation device, to the ablation system and to the methods.

The dilatable member may comprise a tubular wire frame structure coaxially surrounding a distal section of the sheath, wherein a distal end of the tubular wire frame structure is fastened to the tubular member and a proximal end of the tubular wire frame structure is connected to the mechanical control mechanism, such that the proximal end of the tubular wire frame structure is axially displaceable by the mechanical control mechanism.

The wires of the wire frame structure may be made from a shape memory material such as Nitinol, or copper or iron based alloys, or from another flexible and stiff material, such as stainless steel or PEEK or any other suitable material.

The distal end of the tubular wire frame structure may in particular be fastened to or by means of a ring arranged on the tubular member of the sheath at a position close to the distal end of the tubular member.

The proximal end of the tubular wire frame structure may in particular be connected to the mechanical control mechanism by means of a ring slidably arranged on the tubular member of the sheath.

The dilatable member may further comprise a dilatable membrane arranged on the tubular wire frame structure. Thereby, when the proximal end of the tubular wire frame structure is displaced towards the distal end, both the wire frame structure and the dilatable membrane will be dilated and take on a suitable form as discussed above, such as a form selected from the group consisting of a torus, a torus section, a sphere, a sphere section, a cylinder, a cone section, and a pear-like shape.

The mechanical control mechanism may comprise a tubing coaxially surrounding the elongate member of the sheath. In other words, the tubing can be displaced (by sliding it) along the surface of the sheath.

Next, further exemplary embodiments of the ablation system will be explained. However, these embodiments also apply to the sheath, to the ablation device and to the method.

The ablation system comprises a fluid source, such as a container or a bag filled with a saline solution, optionally a saline solution containing a contrast agent, with a connector for connecting with a proximate end of the ablation device, such that the fluid can be supplied to the dilatable member via the canal structure by means of the pump.

The fluid source and the pump may be constituted as a syringe comprising a cylindrical container filled with a predetermined amount of fluid (corresponding to the desired size of the dilatable member in its dilated state) and a plunger for pushing the fluid out of the container and injecting it into the canal structure of the sheath.

The ablation system may comprise a valve, in particular a pressure limiting valve, for limiting the amount of dilation of the dilatable member. The pressure limiting valve may in particular assure that the size of the dilatable member is limited and that the dilatable member is prevented from breaking due to excess filling with fluid.

Hereinafter, the invention will now be described in further detail by way of reference to examples of embodiment to which the invention is, however, not limited.

### BRIEF DESCRIPTION OF THE DRAWINGS

FIGS. 1A-1C show side views of an ablation device according to an exemplary embodiment in three different states of operation.

FIG. 2 shows a cross-sectional view of the ablation device shown in FIG. 1A.

FIG. 3 shows cross-sectional views of sheaths according to three exemplary embodiments of the invention.

FIG. 4 shows a sheath for an ablation device including a handle according to an exemplary embodiment.

FIG. 5 shows an ablation device according to an exemplary embodiment.

FIGS. 6A and 6B show side views of an ablation device according to an exemplary embodiment in two different states of operation.

FIG. 7A shows an axial front view of an RF ablation device according to an exemplary embodiment.

FIG. 7B shows a side view of the ablation device shown in FIG. 7A.

FIG. 8 shows an ablation system according to an exemplary embodiment.

FIG. 9 shows an axial front view and a side view of an ablation device according to an embodiment.

FIG. 10A shows a side view of an ablation device according to an exemplary embodiment.

FIG. 10B shows a side view of a modification of the ablation device shown in FIG. 10A.

FIGS. 11A and 11B show side views of an ablation device according to another exemplary embodiment in two different states of operation.

FIG. 12 shows a side view and an axial front view of a sheath for an ablation device according to an embodiment.

FIG. 13 shows a side view and an axial front view of a sheath for an ablation device according to an embodiment.

FIGS. 14A and 14B show side views of an ablation device according to another exemplary embodiment in two different states of operation.

### DETAILED DESCRIPTION OF EXEMPLARY EMBODIMENTS

The illustrations in the drawings are schematic. In different drawings, similar or identical elements are provided with the same reference signs.

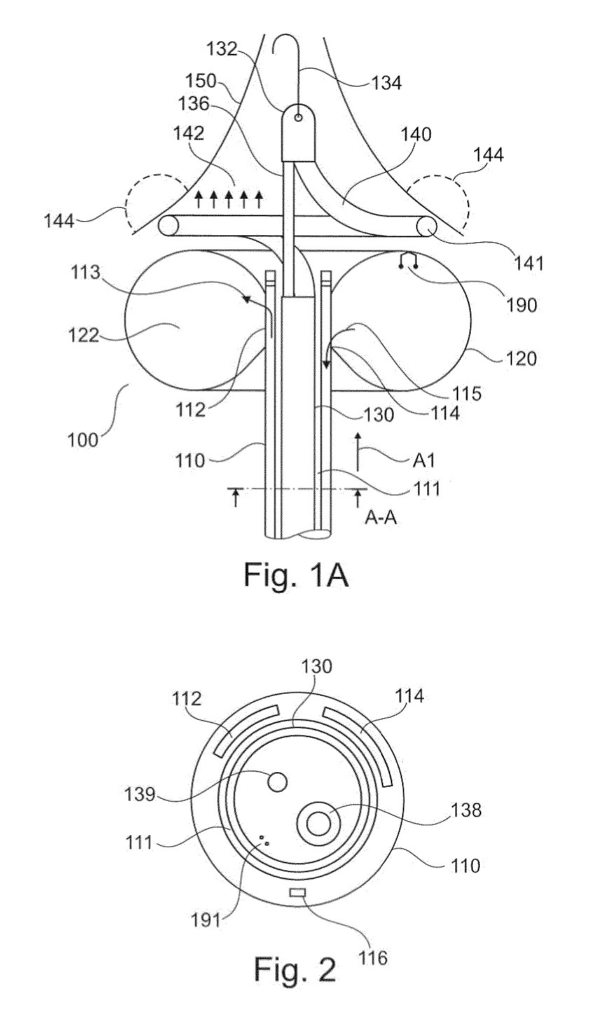

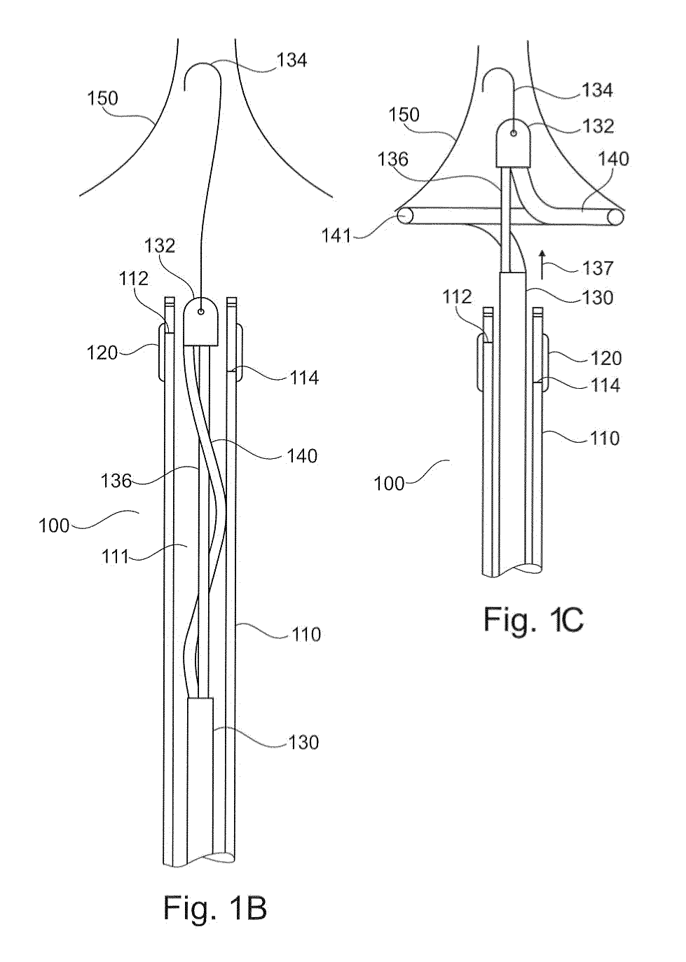

FIG. 1A shows a side view of an ablation device 100 according to an exemplary embodiment in a state of operation where ablation of tissue is performed. The ablation device 100 according to this embodiment is, as discussed in further detail below, a cryoablation device. However, the features, characteristics and advantages discussed in the following are equally applicable to ablation devices relying on different ablation techniques, such as heat or RF (electricity).

More specifically, the ablation device 100 comprises a sheath having an elongate tubular member 110, which defines (i.e. surrounds) an inner lumen 111 in which an ablation catheter 130 is slidably arranged. A dilatable member 120 is arranged at the distal end section (upper end section in the orientation of FIG. 1A) of the elongate tubular member 110 of the sheath. The dilatable member 120 is filled with a fluid 122 (such as a saline solution, optionally mixed with a contrast agent) and surrounds the distal end section of the elongate tubular member 110 circumferentially. The fluid 122 is supplied to the dilatable member 120 through (part of) a canal structure formed in an outer wall of the elongate tubular member 110 and can be discharged through (the same or another part of) the canal structure. More specifically, FIG. 1A shows a canal structure comprising a supply canal 112 for supplying (as indicated by arrow 113) the fluid 122 to the dilatable member 120 in order to dilate it and a discharge canal 114 for discharging the fluid 122 (as indicated by arrow 115) from the dilatable member 120 once the ablation has been conducted in order to allow withdrawal of the ablation device 100.

The ablation catheter 130 comprises an ablation applicator 140 extending through a distal end opening of the elongate tubular member and having the form of a loop 141 for contacting and transferring an extreme cold to the tissue to be ablated. The ablation catheter further comprises a tip 132 in which the end of ablation applicator 140 is fastened and through which a guiding wire 134 for guiding and positioning the ablation applicator 140 also extends. The tip 132 further receives the end of a positioning catheter 136 also extending within the ablation catheter 130. In the state of operation shown in FIG. 1A, the ablation applicator 140 is arranged within a cavity 150, more specifically within the pulmonary vein antrum. The ablation applicator 140 is pushed in the direction of arrows 142 by applying a suitable axial pressure on the sheath (in the direction indicated by arrow A1) while maintaining the position of the ablation catheter, which pressure is then transferred to the loop 141 by the dilatable member 120. Thereby, the loop 141 is brought into firm contact with the tissue that is to be ablated as indicated by the dotted lines 144. In this configuration, the dilatable member 120 furthermore provides an effective isolation such that warm blood located on the side of the dilatable member that is opposite to the ablation applicator 140 is prevented from flowing into the cavity 150 where it could have a negative impact on the effect of the extreme cold applied by the loop 141.

When performing ablation in an atrial chamber of the heart, the ablation applicator 140 may be positioned in a location which is antral from the ostium of the vein or at the ostium of the vein.

The ablation device 100 furthermore comprises a temperature sensor 190 arranged within the dilatable member 120 in a position close to the ablation applicator 140. The temperature sensor 190 enables monitoring the temperature in the vicinity of the ablation applicator. Thereby, an operator may e.g. be able to determine whether the fluid 122 inside the dilatable member 120 is frozen, in which case the sheath 110 and dilatable member 120 should not be moved or withdrawn. It should be noted that the position of the temperature sensor 190 is exemplary and that other locations of the temperature sensor 190 may be utilized. Additional temperature sensors may also be utilized, e.g. within the sheath 110, the ablation catheter 130, the ablation applicator 140, and/or the tip 132.

FIG. 2 shows a cross-sectional view of the ablation device along the line AA shown in FIG. 1A. In particular, FIG. 2 shows a cross-sectional view of the elongate tubular element 110 of the sheath containing the ablation catheter 130. As shown in FIG. 2, the elongate tubular member 110 defines the inner lumen 111 in which the ablation catheter 130 is slidably arranged. The fluid supply canal 112 and the fluid discharge canal 114 are formed within the wall of the elongate tubular member 110 and are essentially formed as “curved rectangles” (or ring segments) extending through the wall of the elongate tubular member 110 in the axial direction to allow fluid communication between the dilatable member 120 at the distal end of the sheath and a fluid source, container, pump or other equipment connected to the proximate end of the sheath. Furthermore, a canal 116 is provided for a wire used to adapt the shape of the sheath, e.g. by bending at least a part of the elongate tubular member 110. As also shown in FIG. 2, the ablation catheter 130 comprises lumina 138 and 139 for respectively supplying and discharging a refrigerant fluid, such as nitrogen or nitrous oxide, to and from the ablation applicator 140. Also shown in FIG. 2 is a pair of wires 191 for enabling communication with the temperature sensor 190 discussed above.

FIG. 1B and FIG. 1C show side views of the ablation device 100 discussed above in conjunction with FIG. 1A and FIG. 2 in two states of operation preceding the state of operation shown in FIG. 1A. As shown in FIG. 1B, the guiding wire 134 extending in front of the remaining ablation device 100 is positioned in the cavity 150 where tissue is to be treated by ablation. At this stage, the dilatable member is empty and is thus fitting tightly around the distal end section of the elongate tubular member 110 of the sheath. Furthermore, the ablation applicator 140 is contained within the inner lumen 111 where it is wound around the positioning catheter 136 while extending substantially in parallel therewith. Thus, in this state of operation, the radial extension of the ablation device 100 is minimal in order to facilitate insertion and positioning within the body. Then, as shown in FIG. 1C, the ablation catheter 130 is moved relative to the sheath in the direction of arrow 137 such that the tip 132 is positioned within the cavity 150 and the ablation applicator 140 takes on the loop-shape 141 as discussed above in conjunction with FIG. 1A. By filling the dilatable member 120 with a fluid through the supply canal 112, the dilatable member 120 expands in size and takes on the torus-like shape shown in FIG. 1A. When filling the dilatable member 120 with fluid, it is important that air present within the dilatable member 120 and the canal structure can escape to the outside. Thus, when filling the dilatable member 120, fluid is supplied through one canal while air escapes through another canal. When all air has escaped, i.e. when the canal structure and dilatable member 120 only contains fluid, the escape canal is closed and further fluid is supplied until the dilatable member 120 has expanded to the desired size and shape.

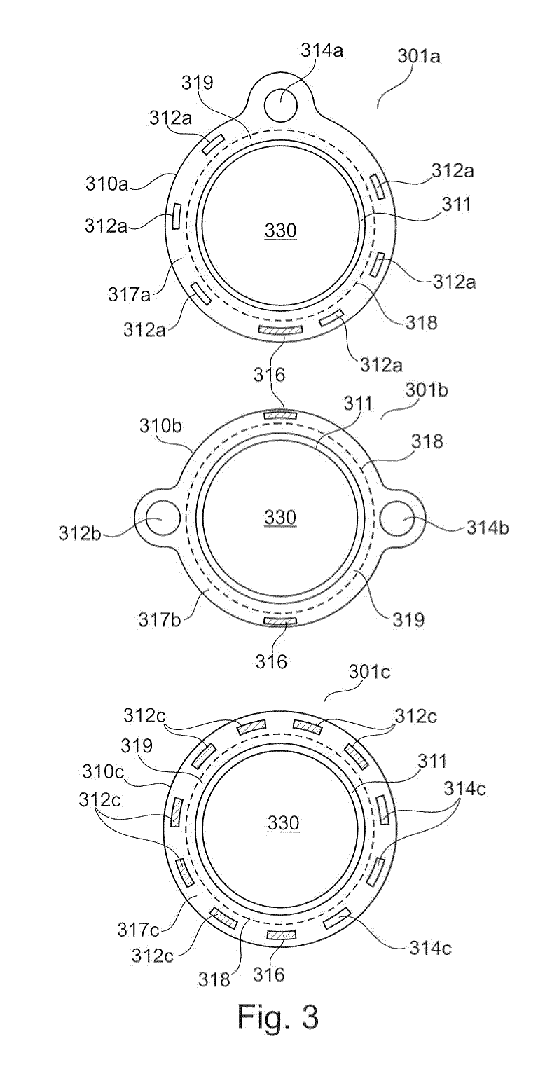

FIG. 3 shows cross-sectional views of sheaths 301a, 301b, and 301c according to three exemplary embodiments of the invention. More specifically, the sheath 301a comprises an elongate tubular member 310a defining an inner lumen 311 in which an ablation catheter 330 is (to be) slidably arranged. The tubular member 310a comprises an outer wall 317a and an inner wall 319 divided by a support structure 318, such as a braiding. The support structure 318 provides reinforcement while the outer wall 317a and inner wall 319 are e.g. made of plastics, such as for example polyamide or polyurethane. Polymers of different durometers can be applied. Durometer can be different for the inner and outer layer but generally will vary also from proximal to distal creating segments. This may tailor the curve of the steerable portion of the steerable sheath to a desired radius and steering angle might be used for adjusting kink stability and rotational stiffness of the shaft structure 310a. A canal structure is formed in the outer wall 317a of the elongate tubular member 310a. More specifically, a number of slit-like, relatively flat (substantially rectangular) fluid supply canals 312a are distributed circumferentially within the outer wall 317a, while a single cylindrical discharge canal 314a is formed opposite from the wire canal 316.

The sheath 301b differs from the sheath 301a discussed above only in that, instead of the multiple flat fluid supply canals 312a, a single cylindrical fluid supply canal 312b is provided opposite from the cylindrical fluid discharge canal 314b. Furthermore, the sheath 301b comprises two wire canals 316 disposed in the outer wall 317b of the elongate tubular member 310b opposite from each other and substantially 90° displaced from the fluid canals 312b and 314b. By having two wires at diametrically opposite positions, the sheath 301b can be bent in two directions.

The sheath 301c differs from the two described above in that no cylindrical canal(s) is (are) provided in the outer wall 317c of the elongate tubular member 310c. Instead, the canal structure only comprises a plurality of slit-like, relatively flat (substantially rectangular) canals distributed circumferentially in the outer wall 317c. More specifically, a first group of canals 312c are used for fluid supply in order to increase the size of the dilatable member (not shown) while a second group of canals 314c are used for fluid discharge in order to decrease the size of the dilatable member.

The skilled person will appreciate that the various supply canals 312a, 312b and 312c described above may equally well be used as discharge canals. Similarly, the various discharge canals 314a, 314b and 314c described above may equally well be used as supply canals. The total cross section of all supply canals may be larger than the total cross sections of the discharge canals.

Such a design takes into account that less cross section is needed for venting air from the balloon (dilatable member) compared to the cross section needed for filling the balloon.

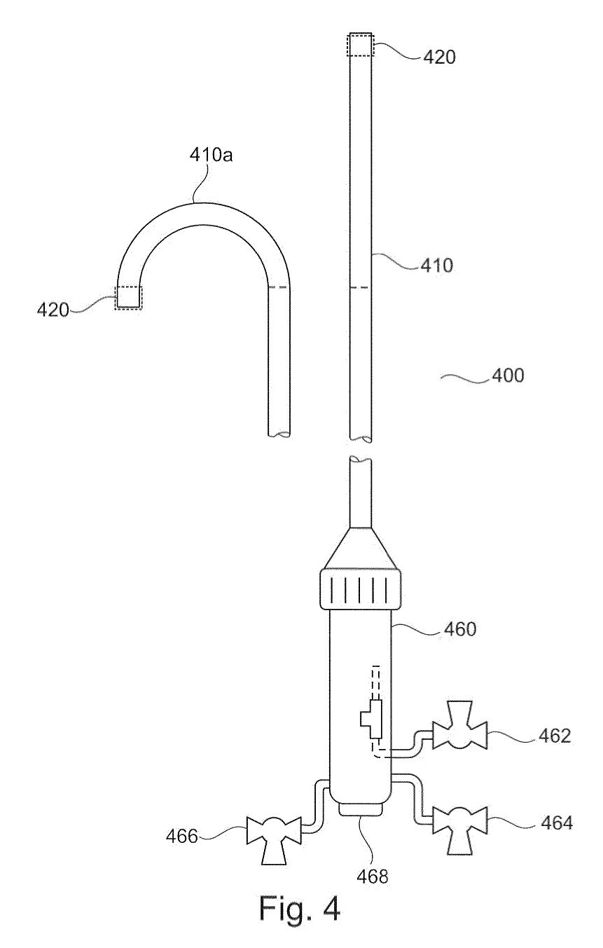

FIG. 4 shows a sheath 400 including a handle 460 according to an exemplary embodiment. The sheath 400 comprises an elongate tubular member 410 and a dilatable member 420 arranged around the distal end section thereof. The elongate tubular member 410 may be bent prior to or during operation and thereby take on the shape shown to the left in FIG. 4 where a section of the elongate tubular member 410a towards its distal end shows a substantially 180° bend. The handle 460 is operable to insert and position the sheath 410, 410a within the body of a patient and to control the ablation process. Furthermore, the handle is connected to valves 462 and 464 for supplying respectively discharging fluid from the dilatable member 420. Also a valve 466 for flushing lumen 111 is shown. A hemostatic valve 468 is foreseen in order to advance the ablation catheter 130 into lumen 111.

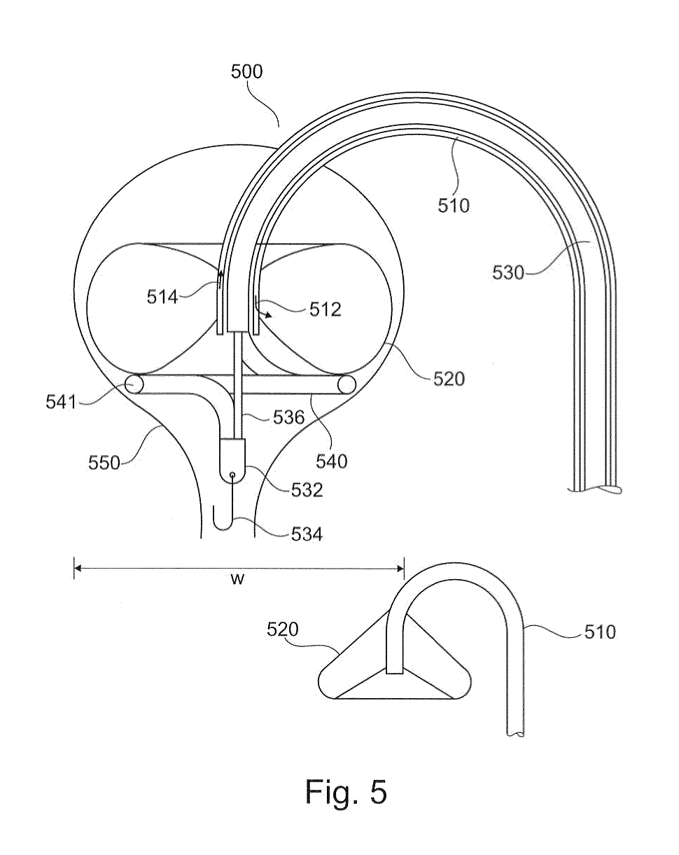

FIG. 5 shows an ablation device 500 according to an exemplary embodiment. More specifically, the ablation device 500 corresponds in most regards to the ablation device 100 shown in FIGS. 1A-1C and discussed above. However, as shown in FIG. 5, a part of the sheath 510 (towards the distal end thereof) is bent. Although the drawing schematically shows a bend of substantially 180°, it should be emphasized that the actual bend will depend on the particular anatomy, and will typically be within a range from 30° to 120° or even more. The ablation device 500 is generally operated in a similar manner as discussed above in conjunction with FIGS. 1 and 2. More specifically, to apply a pressing force on the loop 541 by means of the dilatable member 520, the sheath 510 is moved relative to the ablation catheter 530. The geometry of ablation device 500 fits the anatomic requirements for treating atrial fibrilation in which case the width W of the dilatable member 520 (in its dilated state) may be around 30 mm to 40 mm. For other similar cavities or uses, in particular in the heart, the width W may generally be between 10 and 50 mm. The inner diameter of the sheath is typically between 1.5 and 6 mm, such as between 3 and 5 mm. The lower part of FIG. 5 shows schematically how the dilatable member 520 essentially forms a pillow or pad suitable for pushing the ablation applicator 540 towards the tissue to be ablated.

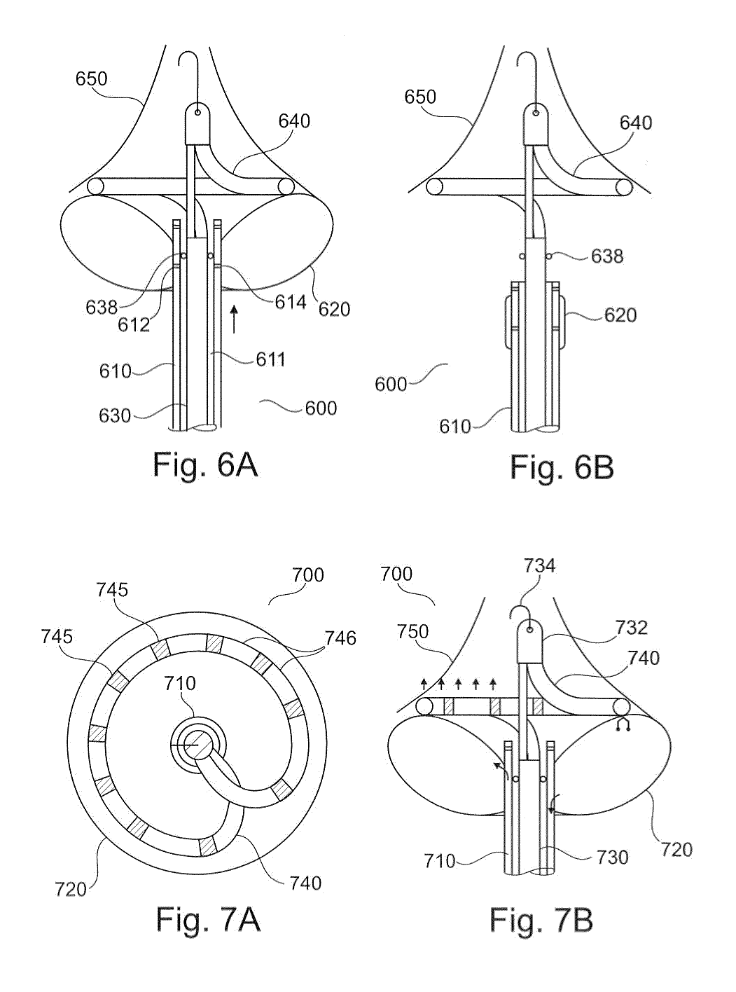

FIGS. 6A and 6B show side views of an ablation device 600 according to an exemplary embodiment in two different states of operation. More specifically, the state shown in FIG. 6A corresponds to the state shown in FIG. 1A and discussed above. That is, the dilatable member 620 is filled with fluid and positioned to push the ablation applicator 640 towards the tissue to be ablated in cavity 650. However, in this embodiment the supply canal 612 does not extend axially within the outer wall of the elongate tubular member 610. Instead, the supply canal 612 is formed as a hole or opening extending radially through the wall of the tubular member 610 such that the inner lumen 611 is in fluid communication with the dilatable member. The return or discharge canal 614 is similar to the canal 114 shown in FIGS. 1A-1C and discussed above. To prevent fluid supplied to the inner lumen 611 from escaping through the distal opening of the elongate tubular member 610, a ring-shaped protruding element 638 acting as a gasket is provided on the outer surface of the ablation catheter 630. FIG. 6B shows a state corresponding to the one shown in FIG. 1C and discussed above, i.e. a state where the ablation catheter has been positioned and the sheath 610 is to be moved towards the ablation applicator 640 and the dilatable member 620 is to be filled with fluid. As shown in FIG. 6B, in this state the ring-shaped protruding element 638 is located outside the elongate tubular member 610 which is consequently not closed at this stage. Thereby, any fluid present in the inner lumen 611 will not be prevented from escaping.

FIG. 7A shows an axial front view of an RF ablation device 700 according to an exemplary embodiment. FIG. 7B shows a side view of the ablation device 700. The configuration of the ablation device 700 is generally the same as the configuration of the ablation device 100 shown in FIGS. 1A-1C and discussed above. However, instead of a cryablation applicator, the ablation device 700 comprises an RF ablation applicator 740 which, as shown in FIG. 7A comprises a plurality of electrodes 745 separated by electrically insulating sections 746. The cross-section view of FIG. 7A is taken from the above (i.e. from the distal front of the device as seen from within the cavity 750) and shows the dilatable member behind the loop-shaped ablation applicator 740.

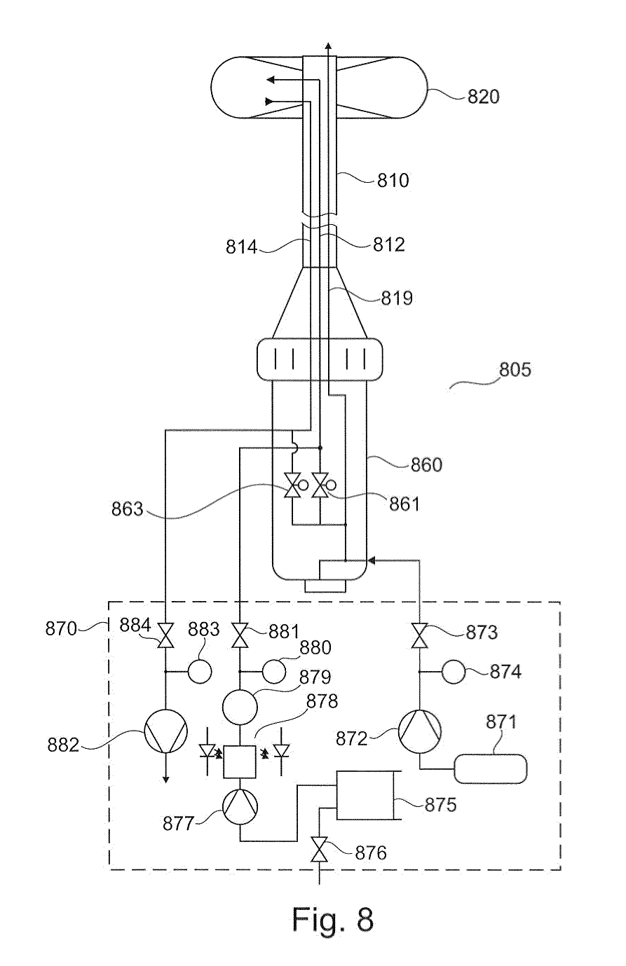

FIG. 8 shows a partial overview of an ablation system 805 (without ablation catheter) according to an exemplary embodiment. The system 805 comprises a handle 860 and a sheath having an elongate tubular member 810 and a dilatable member 820 corresponding to the ablation device shown in FIG. 4. The sheath comprises a canal structure, depicted as supply channel 812 and discharge canal 814. The handle 860 is connected to an external unit 870 comprising a container 871 filled with a saline solution which can be supplied to the handle by pumping unit 872 through valve 873 and monitored by pressure sensor 874. The supplied saline solution can be used to flush the sheath and ablation catheter during positioning as schematically indicated by arrow 819. The external unit 870 further comprises a fluid container 875, preferably containing a predetermined amount V of fluid to be supplied to the dilatable member 820. The fluid container 875 is connected to a valve 876 (for allowing/preventing the fluid from being taken out of the container 875) and a pump for transporting the fluid from the container and into the supply canal 812. Thereby, the fluid passes through an air bubble detecting unit 878, a flow sensor 879, a pressure sensor 880 and a valve 881. The external unit 870 further comprises a vacuum pump 882 connected to the fluid discharge canal 814 through pressure sensor 883 and valve 884. Within the handle 860, an overpressure valve 861 is connected between the fluid supply canal 812 and the flushing canal 819. Similarly, an overpressure valve 863 is connected between the fluid discharge canal 814 and the flushing canal 819. The overpressure valves 861 and 863 serve to prevent breakage of the dilatable member. If the pressure in either canal 812, 814 exceeds a corresponding pressure threshold value, the corresponding overpressure valve 861, 863 will open and allow the fluid to escape into the flushing canal 819.

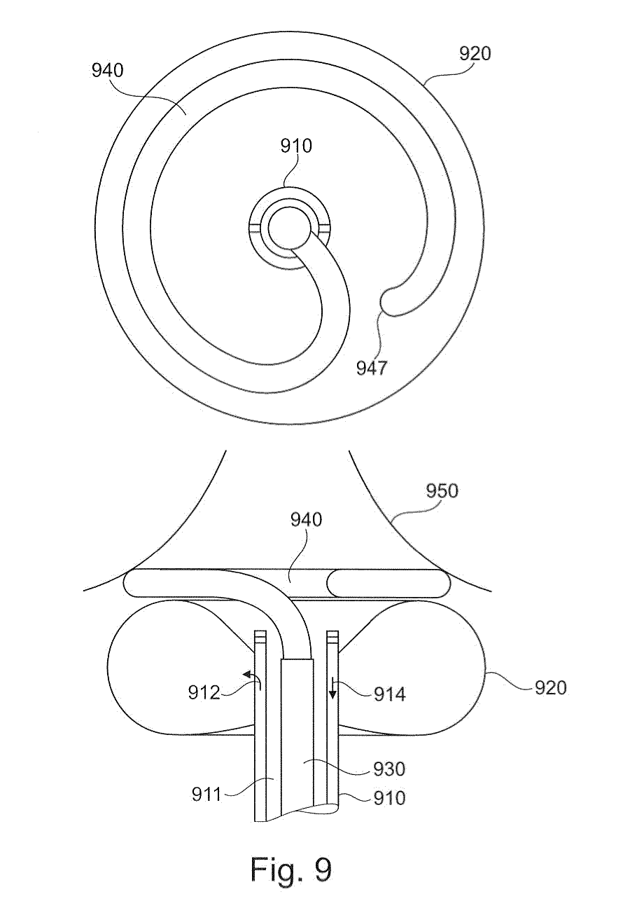

FIG. 9 shows a cross-sectional view and a side view of an ablation device 900 according to an embodiment. The ablation device 900 is essentially configured in the same way as the ablation devices shown e.g. in FIGS. 1A-1C and in FIGS. 5 to 7. However, different from these ablation devices, the ablation device 900 comprises an ablation applicator 940 which does not form a closed loop but instead ends with a tip 947. Furthermore, the ablation device 900 does not comprise an element similar to the positioning catheter 136 shown in FIG. 1A.

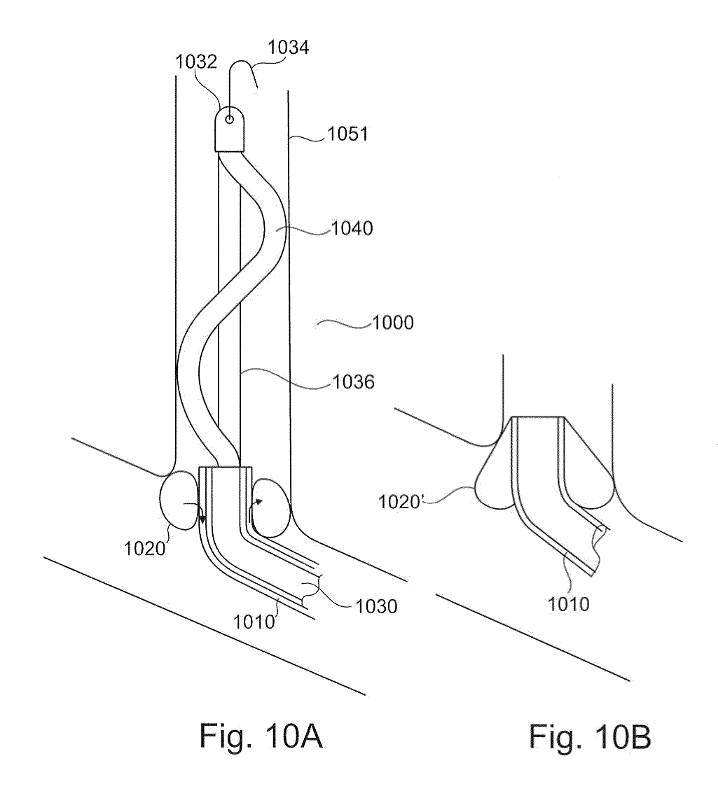

FIG. 10A shows a side view of an ablation device 1000 according to an exemplary embodiment. Like the previously described embodiments, the device 1000 comprises a sheath having an elongate tubular member 1010 and a dilatable member 1020 arranged near the distal end of the tubular member 1010. An ablation catheter 1030 is arranged within the sheath and connected with a helix-shaped ablation applicator 1040 positioned within a cavity 1051, more specifically a blood vessel, either artery or vein. In this embodiment, the dilatable member 1020 has a torus-like shape surrounding the distal end section of the tubular member 1010. The outer diameter of the dilatable member 1020 in the dilated state shown in FIG. 10A may be in the range from 3 mm to 15 mm. In particular, the dilatable member 1020 is positioned to completely block the cavity 1051, thereby preventing warm blood from flowing into the cavity 1051 from the outside.

FIG. 10B shows a side view of a modification of the ablation device shown in FIG. 10A. More specifically, the modification consists in the dilatable member 1020′ having a pear-like shape (rather than the torus-like shape in FIG. 10A), meaning that the outer circumferential surface of the dilatable member 1020′ is tapered towards the cavity 1051, thereby facilitating positioning and fixation within the entrance to or the opening of cavity 1051.

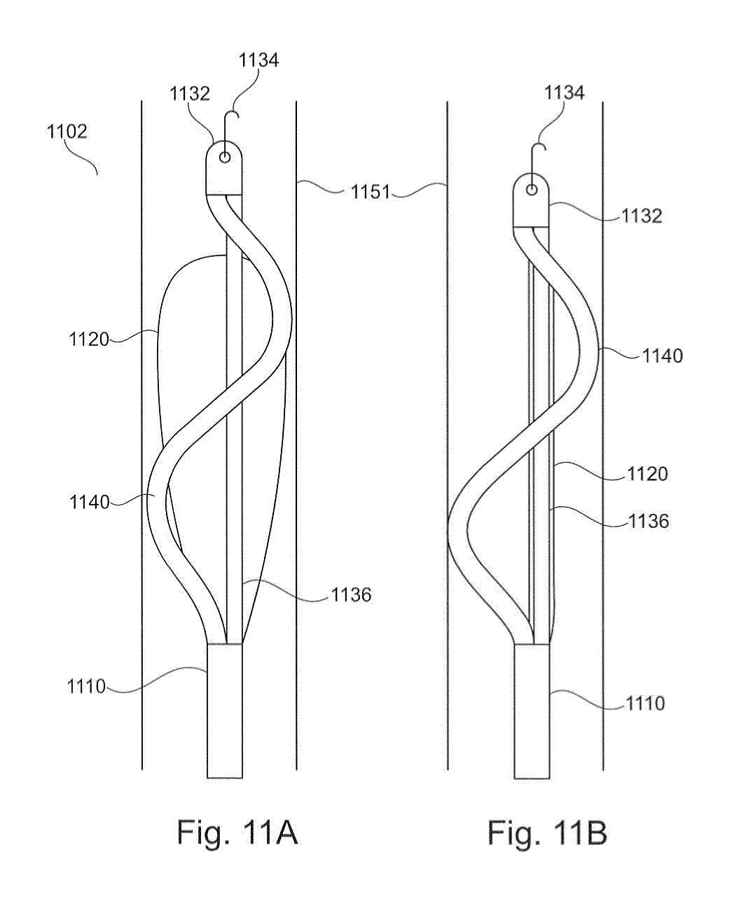

FIGS. 11A and 11B show side views of an ablation device 1102 according to another exemplary embodiment in two different states of operation. First, as shown in FIG. 11B, the ablation device 1102 is positioned such that the helix-shaped ablation applicator 1140 extends within vessel 1151 without however touching the walls of the vessel 1151. The dilatable member 1120 extends in front of the sheath 1110 along with the refrigerant positioning catheter 1136 towards tip 1132. Then, as shown in FIG. 11A, fluid is supplied to the dilatable member 1120 which is expanded and thereby applying an axial force on the ablation applicator 1140 which is consequently pressed against the inner wall of vessel 1151.

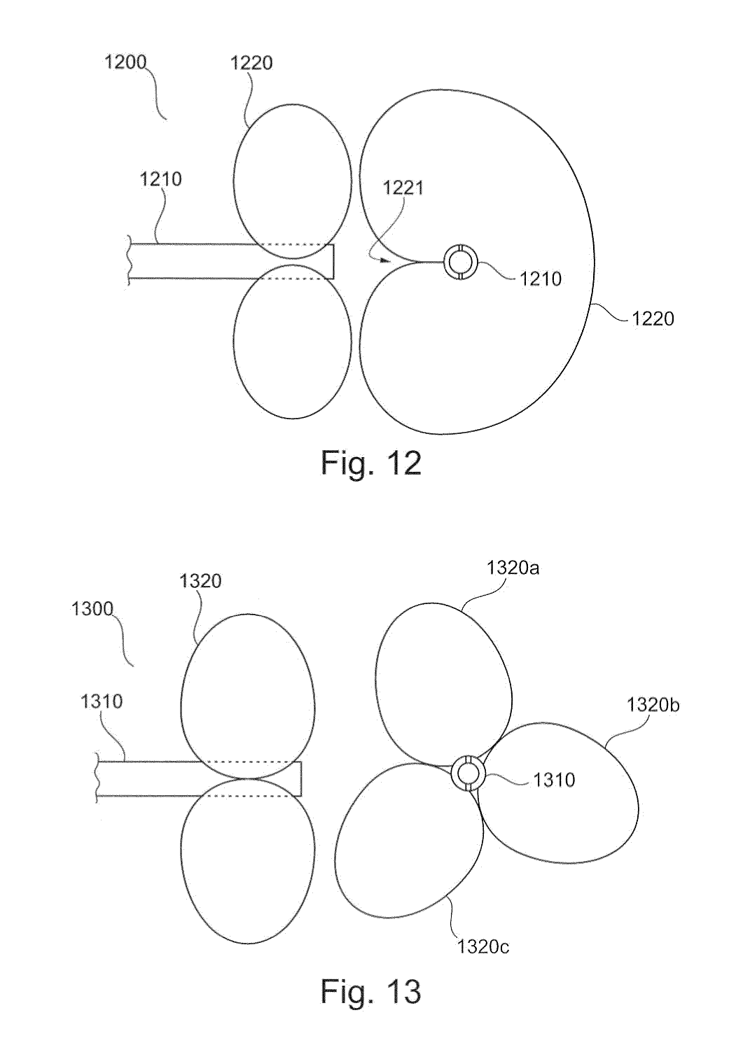

FIG. 12 shows a side view and an axial front view of a sheath 1200 according to an embodiment. More specifically, as shown in FIG. 12, the dilatable member 1220 has a substantially torus-like shape (in the dilated state) with a single “cut-out” or slot 1221. This shape is particularly simple to realize by fastening (gluing) the dilatable member 1220 to the outer surface of the sheath except for an area corresponding to the slit 1221.

FIG. 13 shows a side view and an axial front view of a sheath 1300 according to an embodiment. More specifically, as shown in FIG. 13, the dilatable member 1320 substantially has the shape of a clover or trefoil with three blades 1320a, 1320b, and 1320c (in the dilated state). Also this shape is simple to realize by fastening (gluing) the dilatable member 1320 to the outer surface of the sheath except for three areas corresponding to the slits between the blades 1320a, 1320b, and 1320c.

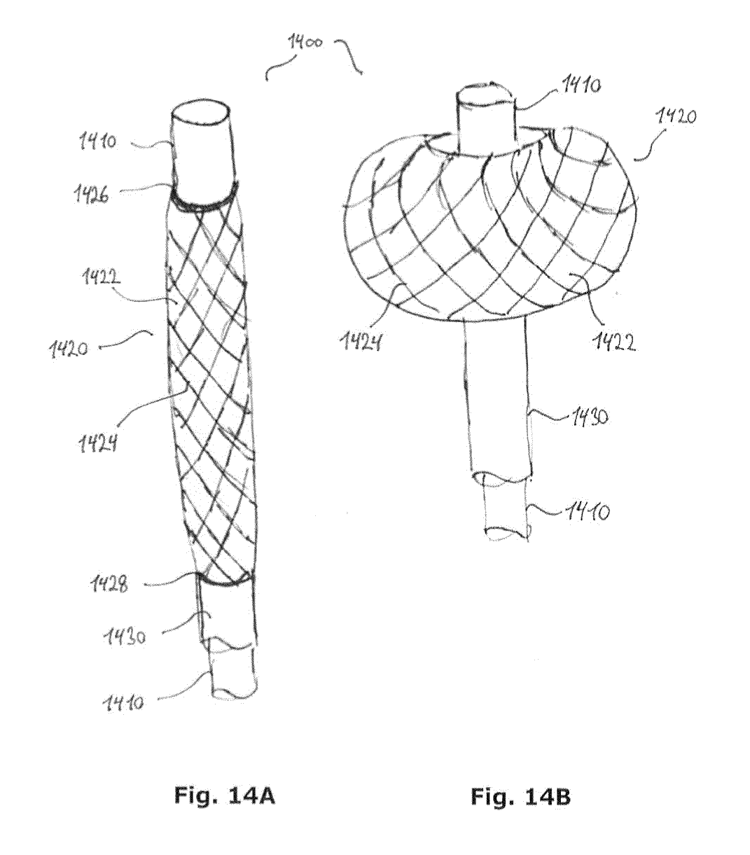

FIGS. 14A and 14B show side views of an ablation device 1400 according to another exemplary embodiment in two different states of operation. In this exemplary embodiment, the dilatable member is not necessarily filled with a fluid (although this may optionally also take place). Instead, a mechanical control mechanism is provided for switching between the non-dilated state shown in FIG. 14A and the dilated state shown in FIG. 14B.

More specifically, in this exemplary embodiment, the dilatable member 1420 comprises a dilatable membrane 1422 that is mounted on a wire frame support structure 1424. As shown in FIG. 14A, the dilatable member 1420 has a tubular shape and is coaxially arranged on the sheath 1410. The wires of the support structure may be made from a flexible and stiff material as for example nitinol, stainless steel or PEEK. On the distal end (the upper end in the drawing), the wires 1424 may be fixed on a ring shaped structure 1426 which is mounted on the sheath 1410 at a fixed position close to the distal end of the sheath 1410. On the proximal end, another ring-shaped structure 1428 is slidably arranged on the sheath 1410. For example, another coaxial tubing 1430 is provided for mechanically displacing the proximal ring-shaped structure 1428 along the main axis of the sheath 1410, thereby acting as a mechanical control mechanism.

For inserting the sheath 1410 into an object (e.g. the human or animal body) the proximal ring 1428 is pulled back to the most proximal position (as shown in FIG. 14A). This forces the wires to be close to the sheath 1410 and thus, the overall diameter of the sheath 1410 with the dilatable member 1420 is small enough to forward the device through and along a vessel to the heart chamber. For expanding the structure, the mechanical control mechanism 1430 is shifted towards a more distal location forcing the wires to span up the dilatable member in a basket like fashion as shown in FIG. 14B. The dilatable membrane 1422 and the wire frame structure 1424 together now form a dilatable member 1420 which can be used for pressing the ablation catheter against the cardiac wall in the same way as described in conjunction with the other embodiments.

In one embodiment, the volume inside the dilatable member 1420 may be flushed for supporting change of shape. Thereby, fluid may be supplied to the dilatable member in any of the ways described above in conjunction with other embodiments. In yet another embodiment, a complete sealing of the inner volume is avoided. This may allow that blood or flushing or body liquid enters the volume inside the dilatable member 1420 during expansion or inflation. In yet another embodiment, the dilatable membrane 1422 may be dispensed with and the dilatable member 1420 is formed only by the wireframe structure 1424.

1. An ablation device, the ablation device comprising
an ablation catheter having an ablation applicator, the ablation applicator having a longitudinal curved shape, and
a sheath comprising an elongate tubular member and a dilatable member arranged at the distal end of the elongate tubular member,
wherein the elongate tubular member defines an inner lumen adapted to receive the ablation catheter having the ablation applicator,
wherein the elongate tubular member comprises an opening at a distal end thereof, the opening being adapted to allow the ablation applicator to extend in front of the distal end,
wherein the elongate tubular member comprises an outer wall in which a canal structure is formed, wherein the canal structure is in fluid communication with the dilatable member, and wherein the canal structure is adapted to supply fluid to the dilatable member in order to dilate it and to discharge fluid from the dilatable member in order to compress it, and
wherein the ablation catheter is slidably arranged within the inner lumen of the sheath.

2. The ablation device according claim 1, wherein the canal structure comprises a supply canal and a discharge canal.

3. The ablation device according claim 2, wherein at least one of the supply canal and the discharge canal is formed as a tubular canal extending within the outer wall in the axial direction of the sheath.

4. The ablation device according to claim 2, wherein at least one of the supply canal and the discharge canal is formed as a substantially rectangular canal extending within the outer wall in the axial direction of the sheath.

5. The ablation device according to claim 1, wherein the canal structure comprises a plurality of supply canals and/or a plurality of discharge canals.

6. The ablation device according to claim 2, wherein at least one of the supply canal and the discharge canal is formed as an opening extending radially through the sheath, thereby bringing the dilatable member in fluid communication with the inner lumen.

7. The ablation device according to claim 1, wherein the dilatable member is arranged at a distal end section of the outer wall, wherein the dilatable member is in particular surrounding the distal end section of the outer wall.

8. The ablation device according to claim 1, wherein the dilatable member is arranged at a distal end section or at a distal tip of an inner wall of the elongate tubular member.

9. The ablation device according to claim 1, wherein the ablation catheter comprises a closing member arranged to block fluid communication between the inner lumen of the sheath and at least a part of the canal structure when the ablation catheter and the sheath are in a predetermined positional relationship.

10. An ablation device, the ablation device comprising
an ablation catheter having an ablation applicator, the ablation applicator having a longitudinal curved shape, and
a sheath comprising an elongate tubular member and a dilatable member arranged at the distal end of the elongate tubular member,
wherein the elongate tubular member defines an inner lumen adapted to receive the ablation catheter having the ablation applicator,
wherein the elongate tubular member comprises an opening at a distal end thereof, the opening being adapted to allow the ablation applicator to extend in front of the distal end,
wherein the sheath further comprises a mechanical control mechanism operable to dilate the dilate the dilatable member and to compress it, and
wherein the ablation catheter is slidably arranged within the inner lumen of the sheath.

11. The ablation device according claim 10, wherein the dilatable member comprises a tubular wire frame structure coaxially surrounding a distal section of the sheath, wherein a distal end of the tubular wire frame structure is fastened to the tubular member and a proximal end of the tubular wire frame structure is connected to the mechanical control mechanism, such that the proximal end of the tubular wire frame structure is axially displaceable by the mechanical control mechanism.

12. The ablation device according claim 11, wherein the dilatable member further comprises a dilatable membrane arranged on the tubular wire frame structure.

13. The ablation device according to claim 10, wherein the mechanical control mechanism comprises a tubing coaxially surrounding the elongate member of the sheath.

14. The ablation device according to claim 1, wherein the dilatable member, in its dilated state, has a shape selected from the group consisting of a torus, a torus section, a sphere, a sphere section, a cylinder, a cone section, and a pear-like shape.

15. The ablation device according to claim 1, wherein the ablation applicator is selected from the group consisting of a cryoablation applicator, a heat ablation applicator, a radio frequency electrical ablation applicator, a microwave ablation applicator, and a laser ablation applicator.

16. The ablation device according to claim 1, wherein the dilatable member is adapted to, in a dilated state, apply a pressing force on the ablation applicator, in particular to apply a pressing force on the ablation applicator in an axial or radial direction of the sheath.

17. The ablation device according to claim 1, wherein the dilatable member is adapted to, in a dilated state, act as an isolating element between the ablation applicator and surrounding tissue and/or fluid.

18. The ablation device according to claim 1, further comprising a temperature sensor for monitoring the temperature in the vicinity of the ablation applicator.

19. (canceled)

20. The ablation device according to claim 1, wherein the ablation applicator has a shape selected from the group consisting of a loop shape and a helix shape.

21.-22. (canceled)

23. A method of operating the ablation device according to claim 1, the method comprising
positioning the ablation catheter and sheath in an object,
dilating the dilatable member by supplying a fluid to the dilatable member through the canal structure,
operating the ablation device to perform an ablation process,
discharging the fluid from the dilatable member, and
removing the ablation device from the object.

24. (canceled)
